(19) United States
(12) Patent Application Publication
(10) Pub. No.: US 2026/0061210 A1
(43) Pub. Date: Mar. 5, 2026

(54) DEVICE AND METHOD FOR MANIPULATING THE NERVOUS SYSTEM BY ELECTRIC FIELDS

(71) Applicant: Zeev Kitov, ROCKVILLE, MD (US)

(72) Inventor: Zeev Kitov, ROCKVILLE, MD (US)

(21) Appl. No.: 19/338,514

(22) Filed: Sep. 24, 2025

Related U.S. Application Data
(63) Continuation-in-part of application No. 18/822,854, filed on Sep. 3, 2024.

(51) Int. Cl. A61N 1/40

(52) U.S. Cl. CPC A61N 1/40

(57) ABSTRACT

A device and method for affecting the patient's neural system by applying an Electric Field generated by an electrode pair electrically floating with respect to the earth potential and skin surface potential of the subject for achieving neurologic effects, such as analgesia, muscle relaxation, stress control, etc. The device includes a housing encasing a power supply, a device printed circuit board, and an electrode printed circuit board supporting at least one electrode pair. Each electrode pair includes an active electrode and a reference electrode arranged in either a non-coplanar or coplanar configuration. When driven by a signal source through a switching circuit and capacitor element, the electrodes generate internal and fringe electric fields that interact with the skin surface to induce lateral currents.

### CROSS REFERENCE TO RELATED APPLICATIONS

This application is a continuation-in-part of a U.S. patent application Ser. No. 18/822,854, filed on Sep. 3, 2024, which is incorporated herein by reference in its entirety.

### FIELD OF INVENTION

The present invention relates generally to neuromodulation, and more particularly to a device and method for manipulating the nervous system using exogenous electric fields.

### BACKGROUND

Stimulation of the nervous system through exogenous electric fields is well known in the art. Conventional methods typically employ electrodes that are isolated from the subject's body, such that the only plausible factor affecting the subject's nervous system is the externally generated electric field. These devices have been used with varying degrees of success in anesthesia, relaxation, sleep enhancement, and the treatment of pain, intractable epilepsy, behavioral disorders, movement disorders, and cardiac arrhythmia. They offer certain advantages over direct electro-contact stimulation, such as eliminating the need for high-quality skin contact, which can be inconvenient to apply.

U.S. Pat. No. 4,915,110, granted to Theri Teck Inc., teaches a pair of electrodes separated from the subject's body by an insulator. An oscillator provides electrical pulses to the electrodes, resulting in the formation of external or fringe electric fields that are applied to selected regions of the body. The device demonstrated effects comparable to non-invasive acupuncture for analgesia, muscle relaxation, and stress control.

U.S. Pat. No. 5,169,380, granted to Brennan, discloses a device for enhancing sleep. The device employs a pair of electrodes positioned on opposite sides of a subject's head, each insulated from the head. The electrodes are supplied with voltages of about 100 V peak-to-peak at frequencies between approximately 5 and 40 Hz.

U.S. Pat. No. 5,782,874, granted to Hendricus G. Loos, teaches both coplanar and capacitor-like non-coplanar electrode structures to form fringe electric fields. The device is intended for relaxation, sexual arousal, and sleep, and is disclosed as being capable of treating medical conditions such as tremors, seizures, panic attacks, and neurological disorders. The patent also discusses a theoretical basis for applying low-frequency, low-intensity electric fields to affect the peripheral nervous system.

Conventional devices rely on fringe electric fields generated by capacitor-like electrode structures that induce small charges and currents in the subject's skin. The electrode structures are separated from the skin by an air gap, which has very high resistance and extremely low capacitance (on the order of picofarads). In addition, the epidermis itself has a substantial capacitance of approximately 1 μF per square centimeter. This combination of the air gap and the epidermis forms a capacitive divider, reducing the effective voltage delivered to the skin by a factor of 10⁹ to 10¹². Consequently, the induced currents in the tissue are on the order of picoamperes per square centimeter, with voltages in the nanovolt range.

Because of these extremely low levels, the induced currents are insufficient to cause classic nerve stimulation by neural membrane polarization. Any therapeutic effect observed from these devices may therefore result from stochastic modulation of spontaneous subthreshold neuronal waves, indirectly affecting synaptic activity rather than directly exciting nerve membranes.

It is also well recognized that the human body incorporates numerous endogenous electrical signals. For example, cardiac action potentials involve voltage transitions from approximately −60 mV to −40 mV during stimulation, and from −40 mV to +10 mV during relaxation. These values far exceed the weak currents induced by conventional electric field devices. In terms of signal-to-noise ratio (S/N), the induced signals represent a condition where S/N is substantially less than 1, making reliable neuromodulation extremely difficult under traditional approaches.

Therefore, in order to achieve meaningful therapeutic or neuromodulatory effects, exogenous electric field stimulation must generate signals strong enough to overcome endogenous electrical noise. Accordingly, there remains a need for improved devices and methods that enhance the efficacy of electric field stimulation of the nervous system.

### SUMMARY OF THE INVENTION

The following provides a simplified summary of one or more embodiments of the present invention in order to facilitate a basic understanding thereof. This summary is not an exhaustive overview of all contemplated embodiments and is not intended to identify essential or critical elements of every embodiment, nor to define the scope of the invention. Its sole purpose is to introduce certain inventive concepts in simplified form, with further details provided in the subsequent description.

A principal object of the present invention is to overcome the disadvantages of conventional systems and methods for manipulating the nervous system through exogenous electric field application.

Another object of the invention is to provide electric fields generated by relatively low-voltage signals that may be applied to localized areas of the body to achieve specific therapeutic effects.

In one aspect, the invention discloses an apparatus and method for generating electric fields that interact with a subject's body by inducing charges and currents into skin tissues without direct electrical contact, thereby producing desired neurological effects.

In one aspect, the disclosed device includes a signal source and at least one pair of electrodes receiving signals from the source to generate electric fields. The signal source may comprise an oscillator or a DC power source. Each electrode pair generates (i) internal electric fields between adjacent electrode surfaces, and (ii) external electric fields between opposite external surfaces that extend beyond the electrode perimeter to form fringe fields. Adjacent surfaces may face each other at angles ranging from 0° to 90°, while opposite external surfaces may define angles ranging from 90° to 360°. The resulting fringe fields extend outside the electrode perimeter to reach and affect the subject's skin tissues. Where alternating current is employed, the external fringe field pulsates at the same frequency as the internal field, both being derived from the same signal source.

The electrode assembly may include multiple electrode pairs, each comprising an active electrode and a reference electrode. Each electrode pair generates a characteristic electric field. Multiple electrode pairs may jointly generate a composite electric field that interacts with the skin surface. In practice, certain cross-field interactions (e.g., between two active or two reference electrodes) may be negligible.

The apparatus thereby produces a combined output electric field composed of spatially joined fields generated by individual electrode pairs. These external fields interact with the epidermis and dermis layers of the skin, inducing charges and currents which in turn produce neurological effects.

The invention further provides a novel electrode assembly that enhances the efficiency of exogenous current induction into skin tissue. In some embodiments, electrode geometry and arrangement may be optimized, such as those described in U.S. patent application Ser. No. 18/822,854, to further improve efficacy.

In another embodiment, efficacy is enhanced by incorporating an external capacitor element in a bypass circuit with the electrode pair. The additional capacitance increases the charge stored between the electrodes, thereby increasing the strength of the induced forces according to Coulomb's law:

$$F = ( {Q}_{1} \times {Q}_{2} ) / {r}^{2}$$

Wherein “Q₁” and “Q₂” are charges and “r” is the distance between them. By increasing electrode capacitance, greater charge storage is achieved, thereby strengthening attraction or repulsion of free charges in tissue. Unlike prior art devices, which rely solely on parasitic capacitance, the disclosed invention employs external capacitor elements to substantially improve efficacy.

In another embodiment, efficacy may be further enhanced by coupling one of the electrodes, preferably the reference electrode, to the physical earth or ground, for example via an AC mains ground terminal.

The earth itself, in interaction with the Heaviside layer, is known to possess an electric charge, producing a geoelectric field on the order of 100 V/m. When one electrode of the pair is grounded, the electrode's field is effectively referenced to earth potential. As a result, the electrode field arrives at the subject's body as electric field pulsations superimposed on the geoelectric field, with the earth's charge participating in current induction into the tissue.

Additionally, the earth has a capacitance on the order of 1,000 μF. By grounding the electrode pair, this capacitance is effectively brought into the electrode field loop, thereby substantially enhancing the amount of charge available to interact with the subject's skin.

In one aspect, different electrode pairs can be driven by signals of different frequencies to target different neurological effects. The signal source may have different output terminals that can receive signals of same or different frequences. Also, more than one signal source can also be used for different active signals.

### BRIEF DESCRIPTION OF DRAWINGS

The accompanying figures, which are incorporated herein by reference, form part of the present specification and illustrate exemplary embodiments of the invention. Together with the detailed description, the figures serve to explain the principles of the invention and enable a person skilled in the art to make and use the disclosed device and method.

FIG. 1 illustrates the overall architecture of the device, according to an exemplary embodiment of the invention.

FIG. 2A is a cross-sectional view of a non-coplanar electrode pair.

FIG. 2B is a cross-sectional side view of a circular coplanar electrode pair.

FIG. 2C is a top view of the circular coplanar electrode pair shown in FIG. 2B.

FIGS. 3A and 3B illustrate electric field patterns generated in the vicinity of skin by a non-coplanar electrode pair (FIG. 3A) and a coplanar electrode pair (FIG. 3B), respectively.

FIG. 4 is a schematic diagram of a non-coplanar electrode pair coupled with a capacitor via a switching circuit, with a DC signal source.

FIG. 5A is a schematic diagram of a pulse amplitude modulation arrangement employing the non-coplanar electrode pair.

FIG. 5B is a schematic diagram of a pulse amplitude modulation arrangement employing the coplanar electrode pair.

FIG. 6A is a schematic diagram of a pulse amplitude modulation arrangement of an exponential electrode signal employing the non-coplanar electrode pair.

FIG. 6B is a schematic diagram of a pulse amplitude modulation arrangement of an exponential electrode signal employing the coplanar electrode pair.

FIG. 7A illustrates a timing diagram of a pulse amplitude-modulated exponential signal.

FIG. 7B illustrates a timing diagram of a pulse amplitude-modulated sinusoidal signal without bias relative to zero voltage.

FIG. 7C illustrates a timing diagram of a pulse amplitude-modulated sinusoidal signal biased with respect to zero voltage.

FIG. 8 is a schematic diagram showing grounding of an electrode pair via an AC mains outlet.

FIG. 9 is a schematic diagram of a more detailed circuit including a grounding arrangement and additional capacitors for enhancing charge induction.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting to embodiments of the invention. As used herein, the singular forms “a”, “an” and “the” are intended to include the plural forms as well, unless the context clearly indicates otherwise. It will be further understood that the terms “comprise”, “comprising,”, “includes” and/or “including” when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

The invention will now be described in connection with certain preferred embodiments with reference to the following illustrative figures so that it may be better understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

### Definitions

Neuromodulation: Neuromodulation refers to the stochastic modulation of spontaneous subthreshold neuronal waves, which may in turn influence normal suprathreshold synaptic activity. Such modulation may affect the function of a target organ or somatic functional system.

Electrodes: The term “electrodes” refers to conductive elements that may be formed as conductive plates, conductive wires (including wiring grids, loops, and pins), or terminals of electronic components.

Reference Electrodes: Reference electrodes are electrodes electrically coupled to a reference potential of the power supply source and receiving the reference signal. The reference potential may correspond to zero voltage of the power supply, a maximum supply voltage, or another stabilized voltage value. Reference electrodes may serve to screen or focus the electric fields formed by the potential difference between active and reference electrodes.

Active electrodes: Active electrodes are electrodes electrically coupled to signal wiring carrying active signals generated by the signal source. The signals may include alternating current (AC), direct current (DC), or combinations thereof (e.g., DC-biased AC signals). The active electrodes may be formed as conductive plates, conductive wires (including grids and pins), or terminals of electronic components.

Capacitor element: A capacitor element refers to a discrete or integrated electronic component having a predetermined capacitance value. This term does not encompass stray or parasitic capacitances inherently present in other devices.

Capacitance unit: A capacitance unit refers to an arrangement including multiple capacitors providing variable capacitance. The capacitance unit enables selection of a desired capacitance from a set of predefined ranges.

Coplanar electrode pair: A coplanar electrode pair refers to two electrodes positioned in the same plane.

Non-coplanar electrode pair: A non-coplanar electrode pair refers to two electrodes positioned in different planes.

It is to be noted that for purposes of the present invention, each area on one side of the human body has a corresponding symmetrical area on the opposite side of the body (e.g., back side versus front side, and right side versus left side), located at approximately the same height or anatomical level. This bilateral and anterior-posterior symmetry may be utilized for placement of electrode pairs or for comparing therapeutic responses across symmetrical body regions.

The present invention pertains to an apparatus and method for the exogenous application of electric fields to a subject's skin, wherein the electrodes do not contact the skin directly, for the purpose of neuromodulation. In one embodiment, the apparatus comprises a signal source and an electrode assembly for generating the electric field. The electrode assembly includes at least one electrode pair, each electrode pair comprising a reference electrode and an active electrode. The reference electrode is electrically coupled to the reference terminal of the power source such that it receives a DC potential equal to the minimum voltage of the power source, the maximum voltage of the power source, or another stabilized voltage value. The active electrode is electrically coupled to the output terminal of the power source, i.e., to wiring carrying active signals generated by the signal source. The active signals may include direct current (DC), alternating current (AC), or combinations thereof (e.g., DC-biased AC signals).

In certain implementations, the electrodes may be formed from conductive plates, wire grids, loops, or terminals of electronic components. The electronic components may include resistors, capacitors, rectifying diodes, Zener diodes, and light-emitting diodes (LEDs). In some embodiments, integrated circuits (ICs) incorporating such components may also serve as electrodes.

In certain implementations, electrodes can be positioned either in a co-planar or non-coplanar way, which depends on their mutual positioning, either in a single plane or in different planes.

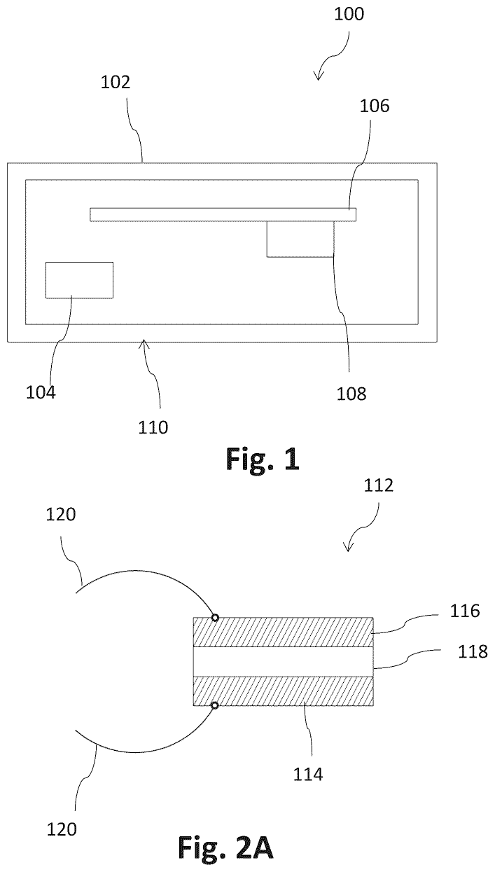

Referring to FIG. 1, there is shown a schematic diagram illustrating an overall architecture of the disclosed device 100. The device 100 is designed to be compact and portable, allowing it to be easily carried and operated by hand. Such portability and compactness enhance user comfort and convenience. Ease of use is further facilitated through the incorporation of integrated printed circuit board (PCB) design, which minimizes bulk and simplifies assembly.

The device 100 includes a housing, which is preferably formed from an insulating material. The housing may be of any suitable size and shape sufficient to accommodate the internal components of the device. In one exemplary embodiment, the housing is generally rectangular in shape with rounded edges and corners for ergonomic handling. In alternative embodiments, other shapes may be employed depending on application-specific requirements. The housing may be fabricated from wood, plastic, metal, or other suitable materials, provided that adequate insulation and safety are maintained.

The housing 102 may encase a suitable power supply 104 for energizing the device 100. In one embodiment, the power supply 104 comprises a battery of appropriate capacity, which may be either disposable or rechargeable. In another embodiment, the power supply 104 may be implemented as an adapter configured to connect to an external power source, such as a household electrical outlet, via conductive wiring. In yet another embodiment, the power supply 104 may utilize wireless power transfer technologies, including but not limited to inductive charging, resonant coupling, or radio frequency (RF) energy harvesting. All such variations are considered within the scope of the present invention.

The device 100 may further include a device printed circuit board (d-PCB) 106 encased within the housing 102. The d-PCB 106 supports and electrically interconnects various electronic components of the device. In addition, an electrode printed circuit board (e-PCB) 108 is also positioned within the housing 102 and is operably coupled to the d-PCB 106. The e-PCB 108 supports the electrode assembly of the device and provides the structural platform for arranging and electrically connecting the electrodes.

FIG. 1 further illustrates a front edge 110 of the enclosure, located along the front wall of the housing 102. During operation of the device for neuromodulation, the front wall of the housing is positioned against a target skin area of the subject such that the front edge 110 is substantially parallel with the tangent of the subject's skin surface. Because the position of the electrode PCB (e-PCB) 108 is fixed relative to the front wall of the housing 102, the electrodes, electronic components, and conductive paths mounted on the e-PCB are likewise fixed in position relative to the subject's skin. In particular, since the front edge of the device PCB (d-PCB) 106 is aligned in parallel with the front wall of the housing 102, the electrodes, wiring traces, and electronic components firmly mounted to the d-PCB at predetermined angles and distances from the front edge maintain a consistent and fixed orientation relative to the subject's skin during use.

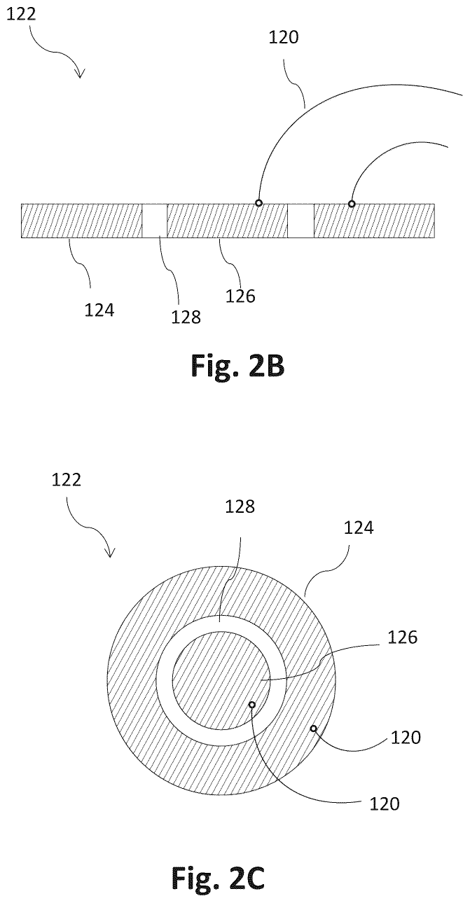

Hereinafter, the term “electrode pair” may also be referred to as an electrode unit of the electrode assembly. Each electrode pair or unit includes an active electrode and a reference electrode. Referring now to FIGS. 2A, 2B, and 2C, there are shown exemplary implementations of the electrode pair.

As illustrated in FIG. 2A, a layer-type electrode pair 112 includes a reference electrode 114 and an active electrode 116, both of which may be formed as metallic plates separated by a dielectric layer 118. The electrodes are arranged in a non-coplanar configuration, such that the reference electrode and the active electrode reside in different planes. Electrical connection between the electrode pair 112 and the power supply may be provided through conductive wires 120

FIGS. 2B and 2C illustrate a coplanar-type electrode pair 122, which includes an active electrode 124 and a reference electrode 126, separated by an insulating layer 128. In this embodiment, both electrodes 124 and 126 are formed as circular, concentric, and co-planar metallic plates. One electrode forms a central disk, while the other is formed as an annular plate surrounding the central electrode. The two electrodes are electrically isolated from one another by an intervening concentric circular insulation 128. It is to be appreciated that the relative positioning of the active and reference electrodes may be varied without departing from the scope of the present invention. Electrical connection between the electrodes and the signal source may be established through conductive wires 120, similar to the arrangement described in connection with FIG. 2A.

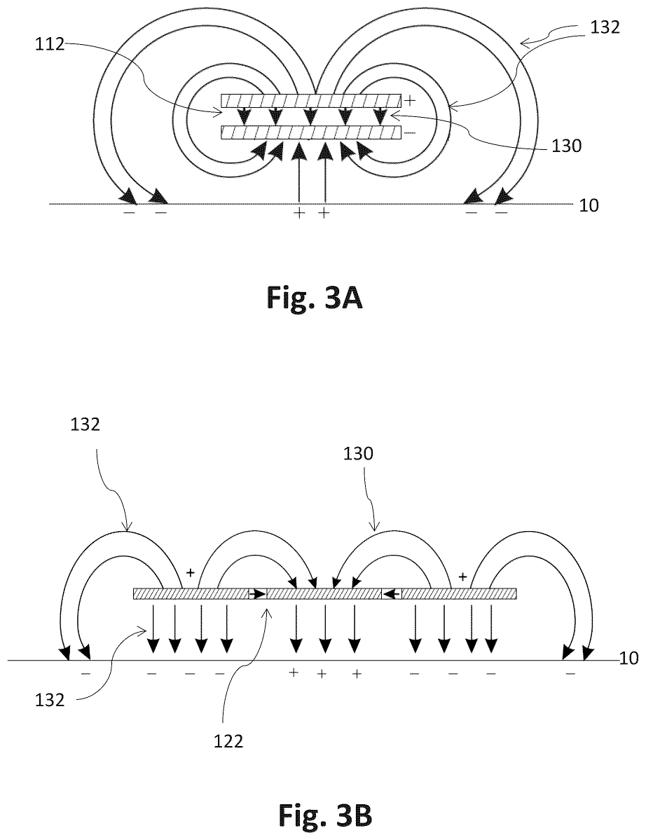

When a potential difference is applied between the electrode plates 114 and 116 of the non-coplanar electrode pair 112, and between the electrode plates 124 and 126 of the coplanar electrode pair 122, electric field (EF) lines are generated, as illustrated by straight and curved vector lines in FIG. 3A (for electrode pair 112) and FIG. 3B (for electrode pair 122). Two types of electric fields are formed: an internal electric field 130, established between the adjacent facing surfaces of the active and reference electrodes, and an external or fringe electric field 132, established between the outer, non-facing surfaces of the same electrodes. The adjacent electrode surfaces generate the concentrated internal field, while the opposite, outward-facing surfaces of the electrodes generate the external fringe fields. The external fringe fields 132 extend beyond the electrode periphery and interact with the skin surface 10, inducing charges of opposite polarity in the skin. This induction results in the flow of lateral currents within the epidermal and dermal tissues, thereby producing neuromodulatory effects.

FIGS. 3A and 3B illustrate the shapes of the electric fields generated by the electrode pairs in the vicinity of the skin 18. In the case of the non-coplanar electrode pair 112 shown in FIG. 3A, the internal electric field is formed between the adjacent facing surfaces of the electrode plates, while the external electric fields are established between the outward surfaces of the electrodes. These external fields exhibit curved, ellipsoidal patterns, portions of which extend into and are partially absorbed by the skin surface. By contrast, in the coplanar electrode pair 122 illustrated in FIG. 3B, the internal electric field is generated between the adjacent edges of the central disk-shaped electrode and the surrounding annular electrode, represented schematically by straight arrows. The flat electrode surfaces in this arrangement produce external fringe fields of semicircular or semi-elliptical form. A substantial portion of these fringe fields is absorbed by the skin surface 18, thereby inducing charges of opposite polarity within the skin tissues.

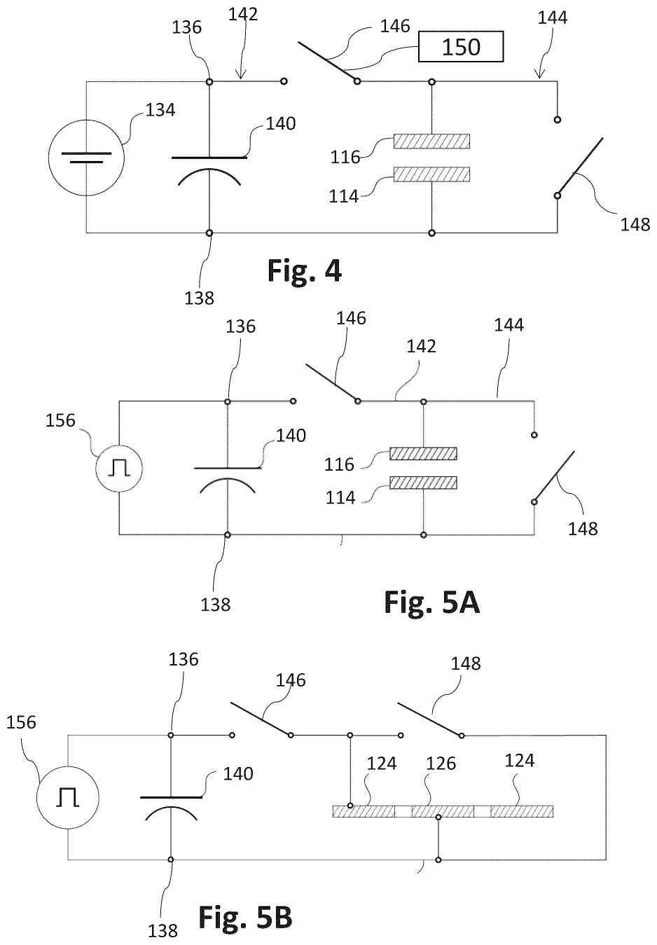

FIG. 4 illustrates an exemplary circuit diagram of the device employing a battery 134 as the signal source, wherein rectangular pulses are applied to the electrode pair comprising the active electrode 116 and the reference electrode 114. The active electrode 116 is electrically coupled to the output terminal 136 of the signal source, while the reference electrode 114 is coupled to the reference terminal 138 of the signal source. A capacitor 140, having a predefined capacitance, is connected across the output and reference terminals. The capacitor increases the charges between the plates and in the outside fringe field thus increasing forces of interaction with free charges in the tissues. The circuit further includes a first path 142 connecting the active electrode 116 to the output terminal 136, and a second path 144 connecting the reference electrode 114 to the active electrode 116. A first switch 146 is provided along the first path, and a second switch 148 is provided along the second path, each switch being operable between an open state and a closed state to selectively interrupt or enable the corresponding current path.

It is to be noted that the first and second switches (146 and 148) cannot be in the closed state at same time. When the first switch is closed, the second switch is turned to the open state. Similarly, when the first switch is open, the second switch is turned to the close state. This may be automatically performed by a switching unit 150. The switching unit 150 can be coupled to at least one or both of the first and second switches (146 and 148). When coupled to the both of the first and second switches (146 and 148), the switching unit can control the opening and closing of the both switches. Alternatively, the second switch can be operably coupled with the first switch so that turning the one switch from one state to another automatically causes the other switch to change its state. In such a case, the switching unit can be coupled to the active switch, and the other passive switch follows the active switch. The switching unit can operate the switches at a predefined frequency. When the switches perform periodical switching, the electrodes of the pair receive pulses of rectangular shape with the frequency as that of the switching unit.

In certain implementations, the capacitance value of the capacitor element 140 may preferably be selected within the range of 0.01 μF to 10 μF. However, the invention is not limited to this range, and other capacitance values are also within the scope of the present disclosure. For example, in some experimental implementations, capacitors having capacitance values up to 470 μF were successfully employed to enhance the efficacy of the electrode pair. Although FIG. 4 illustrates a single capacitor element 140, the number of capacitors employed may vary depending upon design requirements. In certain implementations, multiple capacitors may be coupled between the output and reference terminals of the signal source in parallel configuration through respective switches. For example, as shown in FIG. 9, an additional capacitor 152 may be connected in parallel with the capacitor 140 via a switch 154. The additional capacitor 152 may be selectively connected or disconnected from the circuit by actuating the switch 154, thereby providing variable capacitance capability. In this manner, one or more capacitors can be dynamically activated or deactivated to achieve the desired effective capacitance. Alternatively, a dedicated variable capacitance unit may be employed to provide adjustable capacitance values.

FIGS. 5A and 5B illustrate schematic implementations of the device configured with different electrode geometries. FIG. 5A shows a circuit diagram of the device employing a non-coplanar electrode pair 112, while FIG. 5B illustrates the circuit with a coplanar electrode pair 122. In both configurations, an enhancing capacitor element 140 is coupled across the output terminals of the signal source 156 and further operably coupled to the electrodes of the pair through a switching circuit comprising switches 146 and 148.

The switches 146 and 148 operate in a push-pull mode, such that when the first switch 146 is closed, the second switch 148 is open, and conversely, when the first switch 146 is open, the second switch 148 is closed. When the first switch 146 is closed, the charged capacitor 140 is coupled to the electrodes, and the stored charge produces both the internal electric field between adjacent electrode surfaces and the external fringe field that interacts with the patient's skin. When the first switch 146 opens, the capacitor is disconnected from the electrodes, thereby extinguishing both electric fields. At this moment, the second switch 148 is closed, short-circuiting the two electrodes and discharging any residual charge retained due to stray or parasitic capacitance of the electrodes

The switching circuit is preferably operated periodically at a frequency that is at least one order of magnitude higher than the frequency of the signal generated by the signal source. As a result, the signal produced by the signal source is delivered to the electrode pair in the form of a sequence of high-frequency pulses, with the pulse amplitude following the waveform of the signal source signal. This form of signal presentation is herein referred to as pulse amplitude modulation (PAM).

PAM operation is particularly advantageous when deep penetration of the signal into the subject tissue is desired. Owing to the high carrier frequency of the pulses, the modulated signal is capable of effectively bypassing the capacitive impedance of the epidermis and thereby reaching deeper dermal and subdermal tissues, where it can induce charges and currents for enhanced neuromodulation efficacy.

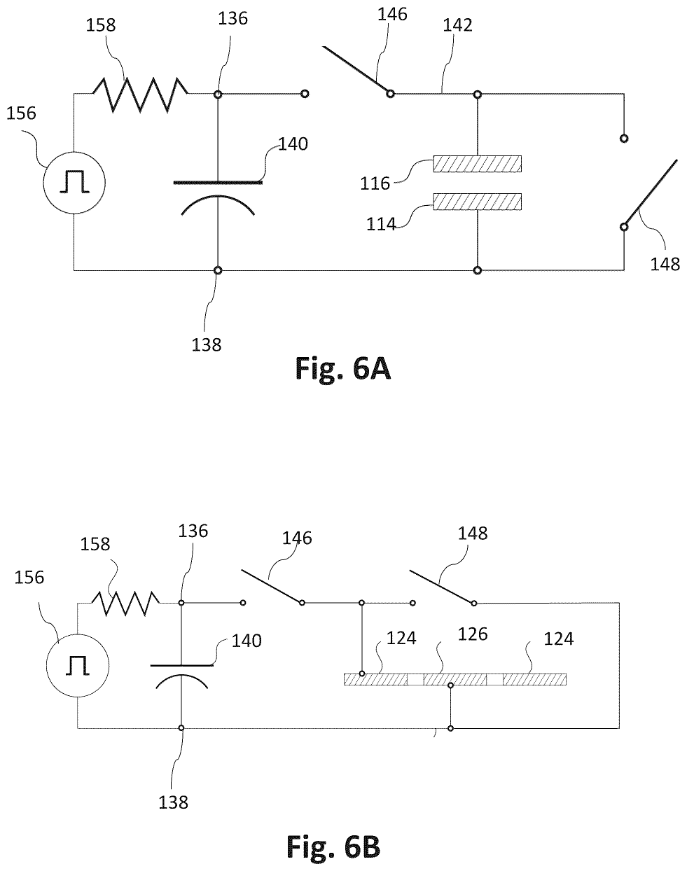

FIGS. 6A and 6B illustrate exemplary circuit diagrams configured to generate exponential-shaped pulses subjected to pulse amplitude modulation by the switching circuit. FIG. 6A corresponds to the implementation using the non-coplanar electrode pair 112, while FIG. 6B corresponds to the implementation using the coplanar electrode pair 122.

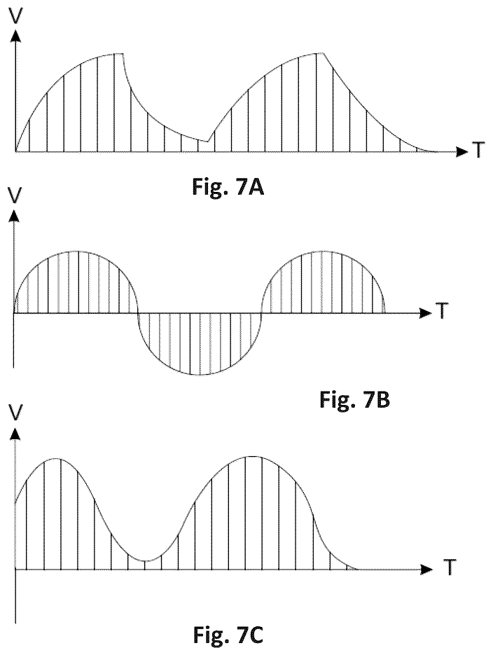

In both implementations, the signal source 156 generates a series of square-wave pulses, which are subsequently shaped into exponential waveforms by a low-pass RC circuit comprising a resistor 158 and the capacitor 140. The resulting exponential pulses are then chopped by the switching circuit including the complementary switches 146 and 148. Consequently, the pulses delivered to the electrode pair (e.g., electrodes 114 and 116) are in the form of high-frequency bursts whose amplitudes follow the exponential decay curve, as illustrated in FIG. 7A.

Additional examples of pulse amplitude modulation are depicted in FIGS. 7B and 7C. In these figures, sinusoidal waveforms are modulated by the switching circuit, with FIG. 7B showing a sinusoidal carrier unbiased with respect to the zero-voltage level, and FIG. 7C showing a sinusoidal carrier biased relative to the zero-voltage level.

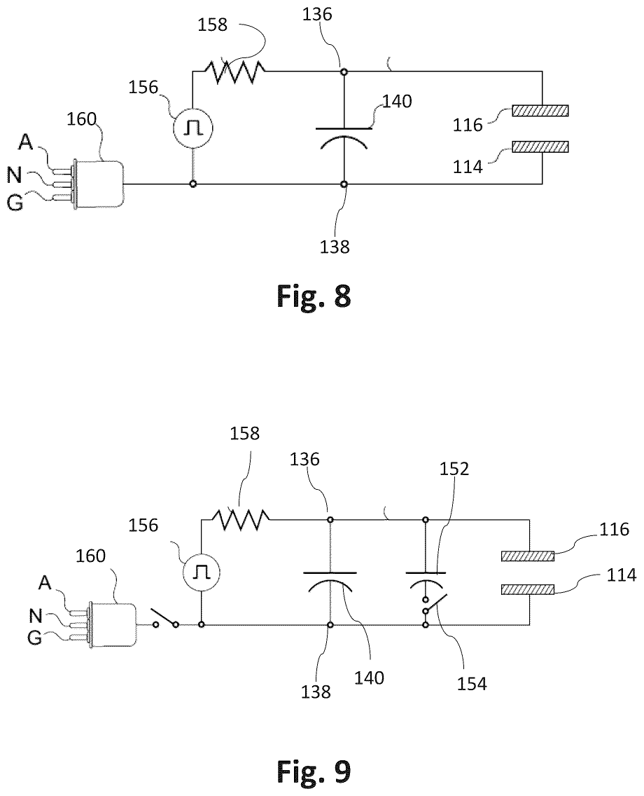

FIG. 8 is a schematic diagram illustrating an exemplary implementation of physical grounding of the electrode pair. As shown, one of the electrodes of the pair—preferably the reference electrode 114—is electrically coupled to the physical ground of the earth via the ground terminal G of an AC mains outlet 160. Consequently, the ground wiring of the device is tied to the physical earth potential, which is generally considered to be zero voltage. It is recognized that all living organisms exist within the geoelectric field of the earth. Relative to its surrounding gaseous atmosphere, the earth has a negative potential, producing an electric field with a voltage gradient of approximately 100 V/m. Thus, the body of a human or animal is continuously exposed to an electric field exceeding 100 V across its length. Moreover, the earth possesses an effective capacitance of approximately 1,000 μF. When a conductive object is physically grounded, it receives an injection of electrons. In ordinary circumstances, humans are insulated from the earth by footwear, leaving their body potential electrically floating. Similarly, the portable device of the present invention is normally ungrounded, and therefore its electrodes are electrically floating with respect to the earth unless intentionally grounded.

When one electrode of the pair is physically grounded—e.g., directly coupled to the earth—a new electrical pathway is established between the earth and the electrodes. As a result, the electrodes are no longer floating but instead assume zero potential relative to the earth. Under these conditions, the electrical parameters of the earth, including its capacitance on the order of 1,000 μF, are effectively integrated into the electric field of the electrode pair. Without being bound by theory, it is believed that this configuration introduces a significant earth-sourced electric charge into the field of the grounded electrode pair.

Consequently, the electric field produced by the electrode voltage interacts with the human subject as a pulsation superimposed upon the natural earth electric field (approximately 100 V/m). Empirical studies conducted on human subjects support the hypothesis that grounding the electrodes enhances the efficacy of the device.

Physical grounding may be selectively implemented by coupling the reference electrode to the ground terminal of the AC mains outlet via an auxiliary switch, as shown in FIG. 9. The switch allows the user to optionally engage grounding, thereby selecting whether and when the additional earth-sourced charge influences the subject's skin.

In certain implementations, the signal source generates AC wave pulses at a frequency of approximately 8 Hz. This frequency coincides with the Schumann resonance, an electromagnetic phenomenon first predicted by Schumann and later experimentally observed as the natural resonance of the Earth-ionosphere cavity excited by global lightning activity. Although the Schumann resonance exhibits extremely low power, human beings are continuously exposed to it throughout life and may be physiologically responsive to this frequency. Furthermore, 8 Hz lies within the alpha brainwave band (8-12 Hz), which is associated with states of wakeful relaxation. Based on these considerations, an 8 Hz signal may serve as a mildly stimulative or relaxation-inducing frequency in the present invention.

In another embodiment, the device generates signals at a frequency of 1 Hz. The 1 Hz frequency lies within the delta brainwave range (1-4 Hz), which is associated with deep sleep, meditation, stress reduction, and unconscious states. Additionally, 1 Hz corresponds to the natural pacing frequency of the human heart muscle, which generates rhythmic pulsations detectable even at peripheral sites of the body, such as the legs.

Experimental results indicate that the application of signals at or around 1 Hz produces calmative effects in human subjects, consistent with the association of delta brainwaves and cardiac pulsation with relaxation and restorative physiological states

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. An electronic device for non-contact induction of charges in a skin area of a subject, the electronic device comprising:
a signal source having an output terminal and a reference terminal; and
a plurality of electrode pairs, each electrode pair comprising an active electrode and a reference electrode, the active electrode being coupled to the output terminal and configured to receive an active signal, the reference electrode being coupled to the reference terminal and configured to receive a reference potential,
wherein each electrode pair is configured to generate an electric field,
wherein at least two of the electrode pairs are configured to receive active signals of different frequencies to generate corresponding electric fields of different frequencies, and
wherein the electronic device is configured to spatially combine the electric fields of the plurality of electrode pairs to form a common output field to apply to the skin area.

2. The electronic device of claim 1, wherein the active electrode and the reference electrode of the electrode pair comprises a conductive plate, wire grid, wire loop, terminal of an electronic component, or any combination thereof.

3. The electronic device of claim 1, further comprising a capacitance element coupled in parallel with the signal source, the capacitance unit having a predefined capacitance value.

4. The electronic device of claim 3, wherein a first current path connects the active electrode to the output terminal, the first current path including a first switch configured to selectively interrupt said path.

5. The electronic device of claim 4, wherein a second current path connects the active electrode to the reference electrode, the second current path including a second switch, wherein the first switch and the second switch are operable in a complementary manner such that both are not simultaneously closed, and wherein the electronic device further comprises a switching unit operably coupled to at least one switch of the first and second switches and configured to periodically switch the said switches at a predetermined frequency, the predetermined frequency being at least one order of magnitude higher than a frequency of the active signal generated by the signal source.

6. The electronic device of claim 5, wherein the signal source generates a square wave signal and the capacitance element is coupled to the signal source through a resistor to form a low-pass RC circuit shaping the waveform into exponential pulses.

7. The electronic device of claim 5, wherein the signal source generates a sinusoidal signal.

8. The electronic device of claim 5, wherein the signal source generates a DC voltage and the switching unit is configured to periodically couple and decouple the signal source output with the electrode pair to produce modulated pulses.

9. The electronic device of claim 1, wherein the reference electrode is selectively coupled to physical ground via a conductor connected to a ground terminal of an AC mains outlet.

10. The electronic device of claim 3, wherein the capacitance element has a capacitance in a range from 0.01 μF to 470 μF.

11. The electronic device of claim 1, wherein the electrode pair is configured as a concentric coplanar arrangement comprising a central circular conductive sheet surrounded by an insulating annulus, and a concentric annular conductive sheet surrounding the insulator.

12. The electronic device of claim 1, wherein the electrode pair is configured as a layered non-coplanar arrangement comprising parallel conductive sheets separated by an insulating dielectric sheet.

13. A method of inducing charges in a skin area of a subject without direct electrode contact, the method comprising:
providing an electronic device comprising a signal source and a plurality of electrode pairs, each electrode pair comprising an active electrode coupled to an output terminal of the signal source and a reference electrode coupled to a reference terminal of the signal source, wherein each electrode pair is configured to generate an electric field, wherein at least two of the electrode pairs are configured to receive active signals of different frequencies to generate corresponding electric fields of different frequencies, wherein the electronic device is configured to spatially combine the electric fields of the plurality of electrode pairs to form a common output field;
generating, by the signal source, the active signal;
applying the common output field to the skin area.

14. The method of claim 13, wherein a capacitance element is coupled in parallel with the signal source, the capacitance unit having a predefined capacitance value.
